(19) United States
(12) Patent Application Publication
(10) Pub. No.: US 2026/0335628 A1
(43) Pub. Date: Oct. 29, 2026

(54) HEART ASSISTANCE OR BACK-UP DEVICE

(71) Applicant: BYPA MEDICAL SOLUTIONS, Dijon (FR)

(72) Inventor: Bertrand DE BROSSES, Dijon (FR)

(73) Assignee: BYPA MEDICAL SOLUTIONS, Dijon (FR)

(21) Appl. No.: 18/997,220

(22) PCT Filed: Jul. 17, 2023
(86) PCT No.: PCT/EP2023/069813

§ 371 Date: Jan. 21, 2025

(30) Foreign Application Priority Data
Jul. 18, 2022 (FR) FR2207327

(51) Int. Cl. A61M 60/531; A61M 60/117; A61M 60/258; A61M 60/441

(52) U.S. Cl. CPC A61M 60/531; A61M 60/117; A61M 60/258; A61M 60/441

(57) ABSTRACT

A device providing temporary extracorporeal circulatory assistance or back-up for a patient's heart, including: a linear actuator configured to move an organ in translation in a chamber in such a way as to cause a pulsatile flow of fluid able to support the activity of said patient's heart, which flow is characterized by a succession of phases of aspiration and phases of ejection of the fluid, a control unit configured to drive the actuator and to control the movement of the organ, which driving is effected while taking into account input data originating from measurements of haemodynamic parameters of the patient, during each phase of ejection said unit moving the organ by a distance.

### TECHNICAL FIELD

An object of the present invention is a device for the assistance or back-up for the heart.

The term assistance is used when part of the blood reaching the heart is withdrawn while the term back-up is used when all, or almost all (>90%), of the blood reaching the heart is withdrawn by the heart's back-up device.

It relates to the technical field of devices used for extracorporeal circulatory assistance/back-up which maintain haemodynamics compatible with life, in the context of cardiac muscle failure, or more generally cardiopulmonary failure, threatening the patient's vital prognosis, following acute or chronic heart failure (coronary insufficiency and/or cardiovascular disease, or other associated pathology).

### PRIOR ART

The heart's function is to distribute blood throughout the organism to transport oxygen and nutrients necessary for the functioning of the various organs and to transport metabolic waste to its CO² elimination organ, the lungs, urea and creatinine to the kidneys. It is divided into two parts, the right heart which receives venous blood (depleted in oxygen) through the vena cava and sends it through the right ventricle to the pulmonary artery to the respiratory system (where it is recharged with oxygen and discharged with CO²), and the left heart which receives oxygenated blood through the pulmonary veins to eject it through the aorta to the various organs. Contractions of the heart muscle (pump) generate a pulsed blood flow in the organism. They allow continuous control of blood flow to the physiological needs of the organism and each organ.

In the presence of a failure of the heart muscle (in the case of a myocardial infarction for example), it is necessary to temporarily back-up the pumping function of said heart muscle with a mechanical circulatory assistance system, the purpose being to guarantee good blood circulation while the heart recovers.

Among the circulatory assistance systems currently used, implantable circulation devices are known, for example described in patent document US2003/032853, but the installation of which is particularly complex and risky. Extracorporeal circulation devices (abbreviated ECC) are also known, which allow to bypass the failing heart by using a servo-controlled pump placed outside the body, which receives blood at the vena cava and injects it at the aorta (thoracic), to mechanically generate a continuous blood flow compatible with the vital needs of the organism, then to proceed with the operation. The main purpose usually sought during circulatory assistance is to regulate the pumped flow in complete match with the physiological needs of the patient, the latter varying continuously.

As a power source, a console or central unit includes a mathematical model designed from physical laws governing the movement of a fluid in a closed circuit. This circuit is generally composed of a pump, a heat exchanger, a flow meter, a blood gas and electrolyte analyser, a pressure sensor as well as biocompatible equipment such as tubing, arterial and venous cannulas, venous reservoir, oxygenator, arterial filter. Usually a centrifugal or peristaltic pump is used as the arterial head pump and four other peristaltic pumps are used for cardiotomy aspiration, cardiac chamber circulation, cardioplegia administration and a back-up pump. If failure persists, ECMO (Extracorporeal Membrane Oxygenation) or ECLS (Extracorporeal Life Support) is indicated. These systems are simpler than a standard ECC and are transportable, the usage time being several days unlike the conventional ECC. Indeed, unlike the conventional ECC, ECMO like ECLS is maintained until the patient's cardiopulmonary recovery or as a relay before a transplant.

Although these systems have demonstrated their effectiveness in their conventional circulatory assistance functions, they are not perfectly satisfactory since the control of the blood flow generated is not optimal with regard to the patient's physiology. The current survival rate for certain pathologies addressed does not exceed 30%. A tired or failing heart will tire even more if the aortic pressure is high during systole: the aortic valves open poorly, the afterload increases and the left ventricular tele-diastolic pressure also increases with an absence of ejection. A leakage of the mitral valve is also observed, which can cause irreversible pulmonary edema. This is one of the main limitations of the prior art systems.

Patent document WO2006/016047 (BERTHIER) describes an extracorporeal circulatory assistance device for the heart provided with a linear motor configured to move a piston in translation in a reservoir in such a way as to cause a blood flow characterised by a succession of phases of aspiration and phases of ejection. The linear motor is servo-controlled to drive the piston in movement, speed and acceleration according to the physiological needs of the patient. However, it has been shown that the match between the pulsatile flow and the physiological needs of the patient is not optimal and does not allow the desired performance to be obtained.

The present invention aims at overcoming all or part of the aforementioned disadvantages. In particular, one purpose of the invention is to propose an assistance or back-up device whose operation allows to maximise the advantages of the assistance or back-up.

Another purpose of the invention is to optimise the real-time synchronisation of the phases of ejection with regard to the patient's physiology in order to maintain haemodynamics compatible with the life of said patient.

Yet another purpose of the invention is to propose an assistance or back-up device whose control is simple to implement and particularly reliable and robust to changes in the patient's physiological parameters.

Also another purpose of the invention is to enable better performance and increased precision of pumping cycles.

### Presentation of the Invention

The solution proposed by the invention is a device providing temporary extracorporeal circulatory assistance or back-up for a patient's heart, comprising:
a linear actuator configured to move an organ in translation in a chamber in such a way as to cause a pulsatile flow of fluid able to support the activity of said patient's heart, which flow is characterised by a succession of phases of aspiration and phases of ejection of the fluid,
a control unit configured to drive the actuator and to control the movement of the organ, which driving is effected while taking into account input data originating from measurements of haemodynamic parameters of the patient, during each phase of ejection said unit moving the organ by a distance D.

The control unit is configured to:
sequence the movement D into a whole number N of cycles Cᵢ, with N>1, each cycle having the same duration Δt,
on each cycle Cᵢ, to calculate a length of movement Lᵢ of the organ such that

$$D = {\sum }_{i = 1}^{N} Li ,$$

which length Lᵢ is calculated according to the input data originating:
   from one or more haemodynamic parameters of the patient that are measured during the cycle Cᵢ, or during one or more cycles preceding said cycle Cᵢ and close to said cycle Cᵢ,
   from the value of a reference pressure Πᵢ based on the Franck-Starling law,
on each cycle Cᵢ, to transmit an instruction to the actuator to move the organ by the calculated length Lᵢ.

On each cycle Cᵢ, the driving of the length Lᵢ allows to optimally adjust the speed of movement of the organ according to the haemodynamic parameters of the patient and, in fact, the pressure of the fluid ejected during said cycle to the physiological needs of said patient. As a result, during each phase of ejection, the pressure of the ejected fluid can be very precisely in line with the Franck-Starling curve in response to changes in the patient's haemodynamic parameters at each cycle Cᵢ. According to the Franck-Starling law, the greater the stretching of the cardiac muscle fibres (produced by the preload), the higher the systolic ejection volume. In other words, the volume of blood ejected per beat will be proportional to the volume of blood contained in the ventricle at the end of diastole.

According to the invention, the ejection pressure is not adapted to a constant target pressure value, but to the actual needs of the patient to find this Frank-Starling mechanism. The device according to the invention thus allows to maximise the advantages of assistance or back-up, while remaining particularly reliable and robust to changes in the patient's physiological needs. The applicant surprisingly found that the survival rate after 15 days of assistance or back-up with the device according to the invention could rise to 80%.

Furthermore, by sequencing the movement of the organ during the phase of ejection and adapting its length of movement on each cycle of the sequence, the pumping cycles have increased accuracy in measurement. Indeed, the value of the ejection pressure on the Frank-Starling curve is controlled at each cycle or, in other words, the patient's responsiveness is controlled at each cycle before introducing fluid into the patient's body. This high-frequency driving (N times higher than the heart rate) ensures an optimal match between the pulsatile flow and the patient's physiological needs.

Other advantageous features of the apparatus that is the object of the invention are listed below. Each of these additional features can be considered alone or in combination with the remarkable features defined above. Each of these additional features contributes, where appropriate, to the resolution of specific technical problems defined further in the description and wherein the remarkable features defined above do not necessarily participate. These additional features can be the subject, where appropriate, of one or more divisional patent applications:

According to one embodiment, the control unit is configured to calculate the travel length Lᵢ according to the following formula:

$${L}_{i} = {L}_{i - 1} + ( {\prod }_{i} - {PR}_{i} - Δ {P}_{cor} ) \times \frac{Δ {t}^{2}}{S \times μ}$$

Where
Lᵢ₋₁ is the length of movement over the cycle Cᵢ₋₁;
Πᵢ is the reference pressure on the cycle Cᵢ;
PRᵢ is the actual fluid pressure measured in the chamber over the cycle Cᵢ;
ΔP_{cor} is a corrective pressure value,
S is the equivalent section of the surface of the organ in contact with the fluid,
μ is a constant.

According to one embodiment, the device includes an ejection cannula implantable in the human body and fluidically connected to the chamber, and wherein the control unit is configured to calculate the length of movement Lᵢ according to the following formula:

$${L}_{i} = {L}_{i - 1} + ( {\prod }_{i} - {PR}_{i} ) \times \frac{Δ {t}^{2}}{S \times μ}$$

Where
Lᵢ₋₁ is the length of movement over the cycle Cᵢ;
Πᵢ is the reference pressure over the cycle Cᵢ;
PRᵢ is the actual fluid pressure measured at the ejection cannula (16);
S is the equivalent section of the surface of the organ in contact with the fluid,
μ is a constant.

According to one embodiment, the control unit is configured to generate, for each phase of ejection, a reference pressure curve according to the Franck-Starling law, the value of Πᵢ corresponding to the value of the pressure on said curve over the cycle Cᵢ.

According to one embodiment, the reference pressure curve according to the Franck-Starling law is identical for each phase of ejection.

According to one embodiment, the reference pressure curve according to the Franck-Starling law is redefined at each phase of ejection or redefined at regular time intervals according to blood pressure values measured at a pre-set frequency, at one end of the patient's body.

According to one embodiment, the control unit is configured to calculate, at each cycle Cᵢ, the reference pressure Πᵢ according to the following formula:

Πᵢ=(PMₛ−Kₛ·VEᵢ₋₁−Rₛ·Qᵢ₋₁)

where:
PMₛ is the maximum systolic pressure whose value is pre-set and/or regulated;
Kₛ is a coefficient comprised between 0.9 and 1.1;
VEᵢ₋₁ is the volume of fluid moved in the chamber during the phase of ejection in the previous cycle Cᵢ₋₁;
Rₛ is a coefficient comprised between 0.4 and 0.6;
Qᵢ₋₁ is the flow rate of fluid ejected from the chamber in the previous cycle Cᵢ₋₁.

According to one embodiment, wherein the number N of cycles Cᵢ is comprised between 45 and 55, and the duration Δt of each cycle Cᵢ is comprised between 0.004 s and 0.006 S.

According to one embodiment, the control unit is configured to dissociate the phases of aspiration and the phases of ejection of the fluid such that during an artificial cardiac cycle, the volume aspirated during the aspiration phase is different from the volume ejected during phases of ejection.

According to one embodiment:
the first cycle C₁ coincides with the natural systole, and starts at the instant when a QRS complex of an ECG signal measured on the patient is detected, or
the first cycle C₁ is delayed relative to the natural systole and starts after a delay comprised between 0.2 and 0.3 seconds from the instant when a QRS complex of an ECG signal measured on the patient is detected.

### BRIEF DESCRIPTION OF THE FIGURES

Other advantages and features of the invention will appear better upon reading the description of a preferred embodiment which follows, with reference to the appended drawings, produced as indicative and non-limiting examples and wherein:

FIG. 1 is a schematic view of the device for temporary circulatory assistance or back-up for a patient's heart according to the invention.

FIG. 2 is a schematic view of a pumping system that can be used in a device according to the invention.

FIG. 3 illustrates the sequencing of organ movement during a phase of ejection.

FIG. 4 illustrates a reference pressure curve according to the Franck-Starling law.

### DESCRIPTION OF THE EMBODIMENTS

The device which is the object of the invention is intended to be used during a degraded haemodynamic situation directly threatening the vital prognosis of a patient 12 (for example with a tissue perfusion pressure—PF—of less than 50 mm Hg). It allows to assist or back-up the heart 13 of the patient 12.

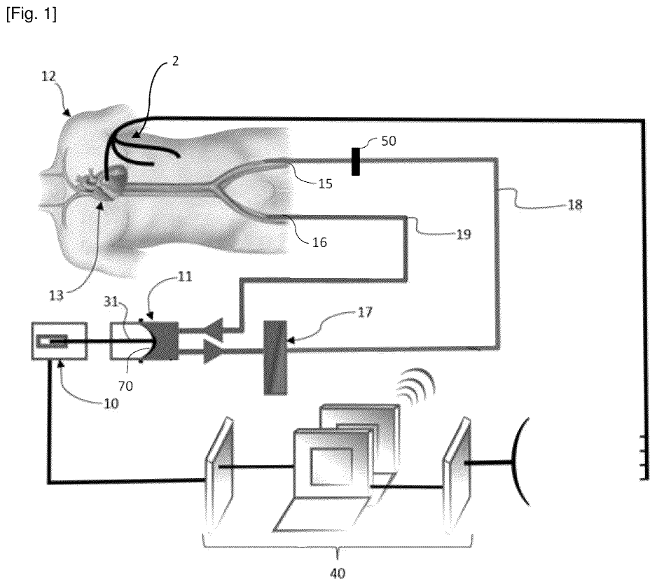

Referring to FIG. 1, the device which is the object of the invention includes a pumping system allowing to partially or totally back-up the heart muscle by aspirating a sufficient quantity of blood during an artificial diastole phase and reinjecting it during an artificial systole phase.

Table 1 below illustrates an example of the sequencing of these phases of aspiration and ejection:

TABLE 1

| Natural heart | Device object of the invention |
|---|---|
| Natural systole | Aspiration |
| Natural start of diastole | Ejection |
| Natural diastole suite | Aspiration |

The sequencing of these phases of aspiration and ejection is described in more detail in the remainder of the description.

According to one embodiment, the operator introduces an intake cannula 15 (for example 21 or 23 French or “Fr”, FRENCH representing ⅓ of a millimetre) capable of drawing blood from the venous system of the patient 12 and an ejection cannula 16 (for example 17 or 19 French) capable of injecting blood into the arterial system of said patient 12. The intake 15 and ejection 16 cannulas that are commonly used on the extracorporeal circulation market (ECC, ECMO, ECLS) are compatible with the invention. Reinforced cannulas are preferably used in order to avoid a collapse of aspiration and/or a kinking of said cannulas which would cause a reduction in the flow.

The operator can perform:
preferably a right heart/left heart setup, of the veno-arterial ECMO type, in the event of failure of the right heart and the left heart, by positioning the intake cannula 15 at a vena cava and the ejection cannula 16 at the aorta. In the latter case, the lungs are also bypassed and an oxygenation system 17 (preferably including a heat exchanger) is placed in the bypass circuit to remove CO₂ from the blood and charge it with O₂ before reinjecting it into the organism. This oxygenation system 17 is advantageously disposed after the reservoir 11, that is to say on the ejection portion 18 of the bypass circuit;
possibly a right heart/right heart setup for partial assistance in the event of right heart failure, by positioning the intake cannula 15 at a vena cava and the ejection cannula 16 at the pulmonary artery.

The cannulas 15, 16 can be inserted percutaneously, in a cardiac and/or vascular catheterisation room or in a resuscitation unit or by a MCAU EMAS unit (Mobile Circulatory Assistance Unit for MCAU and Emergency Medical Assistance Service for EMAS), by introducing them through a peripheral blood vessel and bringing them close to the heart 13, at the targeted veins or arteries. They can also be inserted surgically into a surgical block, mixed implantation percutaneous puncture and surgical opening of the vessels.

These cannulas 15, 16 are connected to the pumping system by catheter-type tubes, also compatible with those usually used for a ECC (for example of ⅜th calibre) to form on the one hand the intake portion 19 and the ejection portion 18 of the bypass circuit.

The device includes a chamber 11 forming a reservoir (equivalent to an artificial external ventricle) and allowing to temporarily store a volume of fluid (for example blood, blood substitute, blood+blood substitute). A linear actuator 10 is configured to move an organ 70 in translation in the chamber 11 in such a way as to cause a pulsatile flow of fluid able to support the activity of the heart 13. This flow is characterised by a succession of phases of aspiration and phases of ejection of the fluid.

As an example and to give an order of magnitude, for a heart beating at 70 bpm (beats per minute), the average flow rate is approximately 5 l/min in ejection and intake. The ejection is brief (high flow rate), the aspiration is slower (approximately half the flow rate). The typical blood volume is approximately 70 cm³ or 70 ml per beat. In 1 min (70 beats), the pumped volume is: 70×70 ml=4900 ml, or 4.9 l. The 70 ml are ejected between 0.2 s and 0.35 s (depending on the patient), so that the maximum instantaneous flow rate on ejection is 270 ml/s or 16 l/min max. This flow rate is approximately half the rate on aspiration. Thanks to the device according to the invention, it is possible to increase (or possibly reduce) the quantities of blood aspirated/ejected to correspond as closely as possible to the actual functioning of a heart and the needs of the organism.

According to one embodiment, the actuator 10 is of the linear motor type or any other equivalent means (for example a cylinder) for moving the organ 70. According to one embodiment, the organ 70 is a membrane fixed, on its periphery, to the internal wall of the chamber 11. It is advantageously made of a flexible and elastic material, elastomer or other. The membrane 70 houses a central insert, not visible in the appended figures, provided with an actuating arm forming a piston 31, this piston being fixed to the actuator 10 by means of a mechanical means of engagement, fixing or connection. According to another embodiment, the organ 70 is in the form of a piston of the type described in the aforementioned patent application WO2006/016047.

According to one embodiment, the actuator 10 is configured to execute the high-velocity movement commands in order to adjust the actual motion of the fluid to said commands. For this purpose, a linear motor with a low time constant (advantageously less than or equal to 10 ms) and capable of generating a significant force (advantageously greater than or equal to 500 N) can be used, to which a lever arm system can be added to accelerate it further. The fluid can then be abruptly moved and stopped at the chosen moment. The physical flow regime of the fluid can thus follow the control signals very closely.

A control unit 40 is provided to automatically control the actuator 10 and drive the movement of the organ 70. This unit 40 can be in the form of a processor, microprocessors, CPU (for Central Processing Unit) integrated into a computer, a calculator or similar means.

Each movement step of the actuator 10 corresponds to an internal volume of the chamber 11. This correspondence between the step of the actuator 10 and the internal volume of the chamber 11 is stored or recorded in a memory area of the unit 40. By controlling the movement of the actuator 10, it is therefore possible to very precisely control the volume of fluid aspirated and ejected by the device during the successive phases of aspiration and ejection.

According to one embodiment, one or more sensors 2 are installed to acquire an electrocardiogramal (ECG signal) which corresponds to an electrical activity of the heart 13. The control unit 40 is configured to drive the movement of the actuator 10 while taking into account input data originating from measurements of haemodynamic parameters of the patient and in particular from this ECG signal. The input data may originate from other measurements of haemodynamic parameters, such as the pressure at the cannula 15 and/or the cannula 16, the oxygen saturation in the blood (measured by means of a pulse oximeter), the variations in pressure or volumes prevailing in an organ or a segment of a limb (measured by means of a plethysmograph), etc. According to a preferred embodiment, the acquisition of these data is carried out in real time, at a fast frequency of 200 Hz.

### Artificial Systole

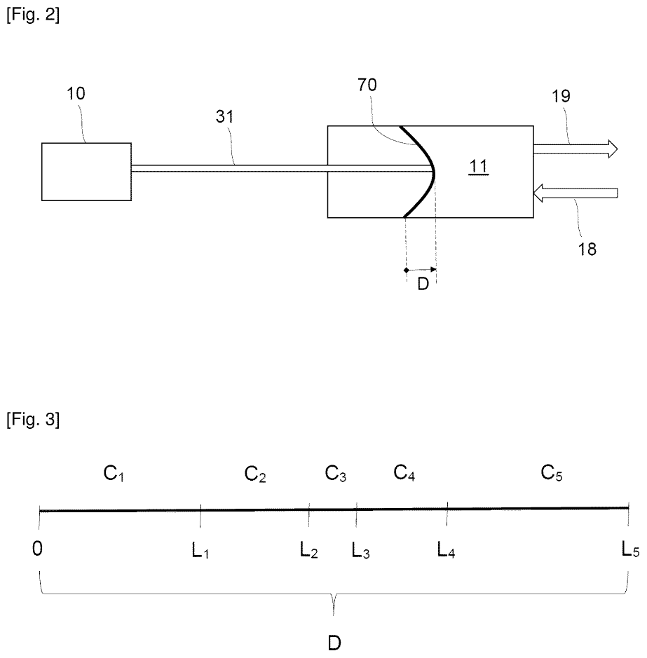

Referring to FIG. 2, during each phase of ejection of the fluid from the chamber 11 (each artificial systole), the unit 40 moves the organ 70 by a distance D. This distance D is not necessarily constant, but can be variable at each artificial systole.

According to one embodiment, the movement D is sequenced into a whole number N of cycles Cᵢ, with N>1, each cycle having the same duration Δt. FIG. 3 illustrates as an explanatory example a sequencing of the movement D into 5 cycles Ci (N=5). The number N is advantageously comprised between 2 and 100, preferably between 10 and 80, and very preferably between 20 and 60. According to a preferred embodiment allowing to obtain an excellent compromise in terms of calculation precision and computing resources required for said calculations, N is comprised between 45 and 55, preferably equal to 50. The duration Δt is advantageously comprised between 0.1 s (second) and 0.003 s, preferably between 0.004 s and 0.006 s, and very preferably equal to 0.005 s. Thus, the unit 40 defines at each cycle Cᵢ the need of the patient 12 and precisely controls the feeding of the organism of said patient according to this same need.

The dynamics of the maximum beat (systole) is of the order of 0.3 s. According to one embodiment, to properly discretise this dynamic, it is divided into at least 20 steps, for example a maximum time step of 0.3/20=0.015 s, corresponding to a minimum frequency of 60 Hz. A good compromise is obtained with a precision margin of 200 Hz, which allows to drive the motions of the organ 70 with a precision of 0.005 s. In particular, this provides an optimal and precise adjustment of the tempos to the 100^{th} of a second.

According to one embodiment, the first cycle C₁ coincides with the natural systole, and starts for example at the time when the QRS complex of the ECG signal is detected. According to another embodiment, the first cycle C₁ is offset relative to the natural systole by an offset of the order of approximately 0.25 seconds (for example between 0.2 and 0.3 seconds) from the time when the QRS complex of the ECG signal is detected. This time offset, made possible by the high calculation rate, allows to ensure that the aortic valve is perfectly closed when the artificial systole is initiated, so that the ventricle is protected from any overload.

Over each cycle Cᵢ, the unit 40 calculates a length of movement Lᵢ of the organ 70 such that

$$D = {\sum }_{i = 1}^{N} Li .$$

In the example of FIG. 3, D=L₁+L₂+L₃+L₄+L₅, with L₁≠L₂≠L₃≠L₄≠L₅. Each length Lᵢ is calculated according to the input data originating from one or more haemodynamic parameters of the patient 12 that are measured during the cycle Cᵢ and a reference pressure value on said cycle Cᵢ. As soon as the length Lᵢ is calculated, the unit 40 transmits an instruction to the actuator 10 to move the organ 7) by said length.

The haemodynamic parameters measured during the cycle Cᵢ may be all or part of those mentioned previously (ECG signal, pressure at the cannulas 15, 16, oxygen saturation, pressure or volume variations, etc.). There is therefore an acquisition of these parameters in each time interval Δt, which allows real-time monitoring of the patient's condition 12 and corresponding driving of the organ 70 which is extremely precise over the distance D.

Furthermore, on each cycle Cᵢ, the unit 40 determines a reference pressure based on the Franck-Starling law.

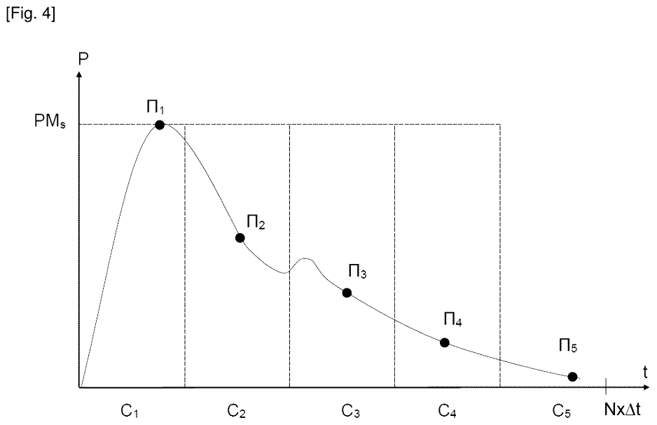

According to a first embodiment, the unit 40 generates, for each phase of ejection, a reference pressure curve according, initially, to the Frank-Starling law. FIG. 4 illustrates a pressure variation curve according to the Frank-Starling law, which typically has the shape of a bell curve passing through a maximum pressure PMs. This reference curve may be identical for each phase of ejection or, preferably, be redefined at each phase of ejection or at regular time intervals (for example every 5 minutes or every 10 minutes), depending on the state of the patient, in particular defined by the measurements of haemodynamic parameters and/or a calculation algorithm based on an artificial intelligence model.

Advantageously, the reference curve is redefined at regular time intervals according to blood pressure values measured at a pre-set frequency, at one end of the patient's body, for example at the tip of a finger. This pressure measurement provides information on the physiological state of the patient 12 and allows the reference curve to be recalibrated. Indeed, the systemic resistances and compliances of the patient's organism change over the course of a day and/or according to any drug treatment of said patient. The artificial pulsatile flow can thus be adapted very simply to these variations. If the pressure measured at the end of the patient's body is lower than a threshold value (for example lower than PMₛ×10%), then the value of PMₛ (for example the peak of the bell curve) is increased. Conversely, if the pressure measured at the end of the patient's body is higher than a threshold value (for example higher than PMₛ×10%), then the value of PMₛ is reduced. This ensures that the fluid penetrates deep into the organism and properly irrigates the patient's organs 12 throughout the duration of circulatory assistance or back-up.

The reference curve is defined over the period N×Δt, where N is the number of cycles Cᵢ and Δt the time interval of each cycle. Each cycle Cᵢ corresponds to a value of the reference pressure Πᵢ, which value is used by the unit 40 as input data for calculating the length Lᵢ. The pressure of the ejected fluid thus follows this reference curve as closely as possible.

According to a second embodiment, the unit 40 calculates, at each cycle Cᵢ, the reference pressure Ili according to the formula [Math 1] based on the Franck-Starling law:

Πᵢ=(PMₛ−Kₛ·VEᵢ₋₁−Rₛ·Qᵢ₋₁) [Math 1]

Where:
PMₛ is the maximum systolic pressure whose value is pre-set and/or regulated by the practitioner or automatically, for example equal to 140 mm·Hg; as explained previously, the value of PMₛ can be redefined at regular time intervals based on blood pressure values measured at a pre-set frequency, at one end of the patient's body;
Kₛ is a systolic coefficient comprised between 0.9 and 1.1;
VEᵢ₋₁ is the volume of fluid moved in chamber 11, during the phase of ejection, in the previous cycle Cᵢ₋₁;
Rₛ is a coefficient expressing the circulatory resistance in the arteries, comprised between 0.4 and 0.6;
Qᵢ₋₁ is the flow rate of fluid ejected from chamber 11 in the previous cycle Cᵢ₋₁.

According to a third embodiment, the reference pressure value Πᵢ is determined by means of a calculation algorithm based on an artificial intelligence model.

According to one embodiment, the length Lᵢ is determined according to the following formula [Math 2]:

$${L}_{i} = {L}_{i - 1} + ( {\prod }_{i} - {PR}_{i} - Δ {P}_{cor} ) \times \frac{Δ {t}^{2}}{S \times μ} \qquad [ Math 2 ]$$

Where
Lᵢ₋₁ is the length of movement on the previous cycle Cᵢ₋₁;
Πᵢ is the reference pressure on the cycle Cᵢ;
PRᵢ is the actual fluid pressure measured in chamber 11 on the cycle Cᵢ;
ΔP_{cor} is a corrective pressure value,
S is the equivalent section of the surface of the organ 70 in contact with the fluid,
μ is a constant (corresponding to blood inertia).

The pressure PRᵢ is advantageously measured by means of a pressure sensor installed in the chamber 11. To provide greater safety to the device, this pressure measurement in the chamber 11 can be correlated with one or more other pressure measurements by sensor installed at the cannula 15 and/or the cannula 16. If one of these different sensors becomes disrupted or defective, it is then possible to stop the pressure regulation method according to the invention and/or to replace said method with another regulation method (for example volume regulation).

The corrective pressure value ΔP_{cor} can be fixed or variable at each cycle Cᵢ. It is for example comprised between 10 mm·Hg and 50 mm·Hg (1 mm·Hg=133.322 Pa). Taking into account in particular the lengths and elasticity of the tubes connecting the chamber 11 to the cannula 16, any apparatuses 17 installed in the bypass circuit, the viscosity of the fluid, the elasticity of the aorta, the pressure PRᵢ measured in the chamber 11 can differ from the actual pressure of the fluid in the aorta. The corrective pressure ΔP_{cor} provides this correction.

The value of μ represents the inertia of the displaced fluid column, which value is for example comprised between 10⁶ kg/m⁴ and 5.10⁶ kg/m⁴.

According to another embodiment, the pressure PRᵢ corresponds to the actual pressure of the fluid measured at the ejection cannula 16 on the cycle Cᵢ. In this case, good results are obtained even by not applying a pressure correction (ΔP_{cor}=0) so that the length Lᵢ can be determined according to the following formula [Math 3]:

$${L}_{i} = {L}_{i - 1} + ( {\prod }_{i} - {PR}_{i} ) \times \frac{Δ {t}^{2}}{S \times μ} \qquad [ Math 3 ]$$

Where
Lᵢ₋₁ is the length of movement on the previous cycle Cᵢ₋₁;
Πᵢ is the reference pressure on the cycle Cᵢ;
PRᵢ is the actual fluid pressure measured at the cannula 16 on the cycle Cᵢ;
S is the equivalent section of the surface of the organ 70 in contact with the fluid,
μ is a constant (identical to that of the formula [Math 2]).

Thanks to the contribution of the invention, the fluid expelled during the artificial systole penetrates deep into the organism of the patient 12, according to his physiological needs, in order to irrigate the different organs. The flow/pressure profile of the ejected fluid combines with the arterial compliances so that the systolic wave propagates correctly in the organism.

The best results are obtained when the acquisition of the haemodynamic parameters, the actual pressures, the determination of the reference pressure, the development of the movement command Lᵢ and its execution by the unit 40 are carried out during the cycle Cᵢ. However, according to another embodiment still giving suitable results, the acquisition of the haemodynamic parameters and the actual pressures, the determination of the reference pressure, the development of the movement command Lᵢ and its execution by the unit 40 can be carried out during one or more cycles preceding the cycle Cᵢ and close to said cycle Cᵢ. Close means three or four cycles preceding the cycle Cᵢ during which the movement command is executed. For example, the acquisition of the haemodynamic parameters can be carried out on the cycle Cᵢ₋₃, the actual pressure of the fluid also measured on the cycle Cᵢ₋₃, the calculation of the reference pressure Πᵢ₋₂ carried out during the cycle Cᵢ₋₂, the development of the movement command Lᵢ carried out on the cycle Cᵢ₋₁ to be executed on the cycle Cᵢ. The person skilled in the art will understand that other sequencing in time is possible.

### Artificial Diastole

The execution of artificial the diastole is independent of that of the artificial systole. According to one embodiment, the intake of blood into the chamber 11 (artificial diastole) is carried out throughout the natural cardiac cycle, apart from the time dedicated to the artificial systole.

According to one embodiment, the time of the artificial diastole is about 0.5 seconds (about ⅓ of the artificial systole), the organ 70 moving about 1 cm.

According to one embodiment, the flow rate of fluid aspirated into the chamber 11 during artificial diastole is high, or even very high, at least 2 to 3 or even 5 litres per minute. Given this high aspiration flow rate, the risk of collapse of the patient's vena cava 12 is high, or even very high. It is therefore particularly interesting to manage the aspiration as finely as possible, that is to say to be able to modulate or modify the aspiration of the fluid taken from the patient's vena cava in order to avoid any risk of collapse.

To achieve this purpose, a pressure sensor is advantageously installed in the intake portion 19 (at the intake cannula 15 or between said cannula and the chamber 11). This pressure sensor has a measurement time constant of less than 20 milliseconds, advantageously less than 10 milliseconds, in order to detect very quickly and very regularly over time the pressure and any modification thereof. The unit 40 can thus modulate or stop the aspiration when the pressure detected by said sensor reaches a threshold pressure value and/or the pressure increases/decreases above or below a threshold pressure acceleration/deceleration slope. According to one embodiment, the threshold pressure value is comprised between 6 mm·Hg and 15 mm·Hg, preferably equal to or substantially equal to 12 mm·Hg. Any risk of collapse at the vena cava is avoided, thanks to the threshold values (absolute and/or acceleration slope) and the continuous and rapid detection (high frequency) of the pressure in the intake portion 19.

The unit 40 has computer resources wherein two types of alerts have been programmed relative to the pressure detected in the intake portion 19. First, a threshold level in absolute pressure value which, if reached, triggers a modification of the blood pumping, typically by reducing the pumping flow rate. Then, the second alert consists of the acceleration/deceleration of the pressure, between two pressure measurements over time: again, if this threshold slope (acceleration/deceleration) is reached, the blood aspiration is modified, typically by reducing or stopping it. Concerning the threshold slope, the predictive program for the evolution of the pressure typically uses the Reynolds number or the Reynolds wave. The Reynolds number corresponds to a dimensionless number that is used in fluid mechanics. This quantity allows to characterise a flow, in particular the nature of its regime. It is thus possible to know whether a flow is laminar, transient or turbulent.

At each pulse, an order of magnitude of the volume of fluid aspirated is of the order of 50 ml (millilitre) but this can go up to 100 ml, depending on the patient. A natural cardiac output is comprised between 2.5 and 4.5 l/mn/m² (litre per minute per square meter). In other words, the larger the surface area of a human body (and therefore its weight), the greater the blood circulation. In practice, to adjust the weight or volume of fluid to be aspirated at each pulse, the doctor considers the weight of the patient 12 and deduces the volume at each pulse.

For a heart beating at 70 bpm (beats per minute), the aspiration time is approximately 0.56 seconds, so that a normal flow rate is approximately 90 ml/s (millilitre per second per systole) or 5 l/min (litre per minute). However, in practice, the doctor tends towards an average flow rate of 3 or 4 l/min. Thanks to the device according to the invention, the aspirated quantities of blood can be increased (or optionally reduced) to correspond as closely as possible to the actual functioning of a heart, without risking collapse at the vena cava.

For example, for the pressure threshold value (first alert) and the pressure slope or acceleration threshold value (second alert):
if the slope or acceleration of the measured pressure is 50% lower than it should be during an aspiration cycle, then this means that there is an imminent risk of collapse, and the unit 40 immediately reduces the aspirated flow;
if the absolute pressure falls below the threshold pressure value of 12 mm·Hg, then the unit 40 immediately stops pumping for this aspiration cycle, because the risk of collapse is very/too significant. Pumping restarts at the next aspiration cycle.

According to one embodiment, on an artificial cardiac cycle, the volume aspirated during the artificial diastole corresponds to the volume ejected during the artificial systole. However, depending on the condition of the patient (for example: hypertension, condition of the venous system, arterial obstruction, blood viscosity, etc.), the volume aspirated during the artificial diastole may be different from the volume ejected during the artificial systole. For example, the unit 40 may control the movement of the organ 70 so that during the artificial diastole, a sufficient volume of fluid is aspirated to ensure good circulation in the patient's body, this aspiration being able to be slow (for example: 70% of the cardiac cycle) and with low dynamics to avoid the risks of collapse. For a patient suffering from hypertension, it may be advantageous not to reinject all of this aspirated volume to avoid contributing to an overpressure of the patient's organism. For example again, for a patient 12 suffering from arterial obstruction, the unit 40 can be configured to perform two artificial diastoles on two successive cardiac cycles (without artificial systole), to control the artificial systole on the following cycle, with an optimal flow/pressure profile so that the systolic wave propagates deep into the organism of the patient 12 despite the arterial obstruction.

More generally, the configuration of the unit 40 allows to dissociate artificial diastoles and artificial systoles: the aspiration of the fluid can be independent of the ejection regime in the aorta, which corresponds to the reality of the patient's organism 12. The chamber 11 combined with the controlled movement of the organ 70, ensures a diastolic balance assignment function (AED): the average volume of the chamber 11 allows to adjust the volume of fluid in the patient's organism 12, and therefore the average blood pressure in said organism.

The best results for achieving optimal synchronisation between artificial circulation and the needs of the patient's organism 12 are obtained in particular by controlling the following two elements:
A fast processing of measurements (preferably with a fast acquisition frequency of 200 Hz or that order of frequency) to send commands to the actuator 10 in real time.
A high velocity command execution to match actual fluid motion to the commands.

The arrangement of the various elements and/or means and/or steps of the invention, in the embodiments described above, should not be understood as requiring such an arrangement in all implementations. In any event, it will be understood that various modifications may be made to these elements and/or means and/or steps, without departing from the spirit and scope of the invention.

Furthermore, one or more features disclosed only in one embodiment may be combined with one or more other features disclosed only in another embodiment. Similarly, one or more features disclosed only in one embodiment may be generalised to other embodiments, even if that feature or features are described only in combination with other features.

In any event, in the claims, any reference sign in parentheses cannot be interpreted as a limitation of the claim.

Another aspect not covered by the invention and the claims, relates to a device for temporary circulatory assistance or back-up for a patient's heart, which is not extracorporeal but implantable and comprising:
a linear actuator configured to move an organ in translation in a chamber in such a way as to cause a pulsatile flow of fluid able to support the activity of said patient's heart, which flow is characterised by a succession of phases of aspiration and phases of ejection of the fluid,
a control unit configured to drive the actuator and to control the movement of the organ, which driving is effected while taking into account input data originating from measurements of haemodynamic parameters of the patient, during each phase of ejection said unit moving the organ by a distance D.

The control unit is configured to:
sequence the movement D into a whole number N of cycles Cᵢ, with N>1, each cycle having the same duration Δt,
on each cycle Cᵢ, to calculate a length of movement Lᵢ of the organ such that

$$D = {\sum }_{i = 1}^{N} Li ,$$

which length Lᵢ is calculated according to the input data originating:
from one or more haemodynamic parameters of the patient that are measured during the cycle Cᵢ, or during one or more cycles preceding said cycle Cᵢ and close to said cycle Cᵢ,
from the value of a reference pressure Πᵢ based on the Franck-Starling law,
on each cycle Cᵢ, to transmit an instruction to the actuator to move the organ by the calculated length Lᵢ.

The other features claimed and described above apply to this embodiment not covered by the invention.

1-10. (canceled)

11. A device providing temporary extracorporeal circulatory assistance or back-up for a patient's heart, comprising:
a linear actuator configured to move an organ in translation in a chamber in such a way as to cause a pulsatile flow of fluid able to support the activity of said patient's heart, wherein the flow has a succession of phases of aspiration and phases of ejection of the fluid,
a control unit configured to drive the actuator and to control the movement of the organ, which driving is effected while taking into account input data originating from measurements of haemodynamic parameters of the patient, during each phase of ejection said unit moving the organ by a distance D, wherein the control unit is configured to:
sequence the movement of distance D into a whole number N of cycles Cᵢ, with N>1, each cycle having the same duration Δt,
on each cycle Cᵢ, to calculate a length of movement Lᵢ of the organ such that
$$D = {\sum }_{i = 1}^{N} Li ,$$
which length Lᵢ is calculated according to the input data originating:
from one or more haemodynamic parameters of the patient that are measured during the cycle Cᵢ, or during one or more cycles preceding said cycle Cᵢ and close to said cycle Cᵢ,
from the value of a reference pressure Πᵢ based on the Franck-Starling law, and
on each cycle Cᵢ, to transmit an instruction to the actuator to move the organ by the calculated length Lᵢ.

12. The device according to claim 11, wherein the control unit is configured to calculate the length of movement Lᵢ according to the following formula:
$${L}_{i} = {L}_{i - 1} + ( {\prod }_{i} - {PR}_{i} - Δ {P}_{cor} ) \times \frac{Δ {t}^{2}}{S \times μ}$$
wherein
Lᵢ₋₁ is the length of movement over the cycle Cᵢ₋₁;
Πᵢ is the reference pressure on the cycle Cᵢ;
PRᵢ is the actual fluid pressure measured in the chamber over the cycle Cᵢ;
ΔP_{cor} is a corrective pressure value,
S is the equivalent section of the surface of the organ in contact with the fluid, and
μ is a constant.

13. The device according to claim 11, further comprising an ejection cannula implantable in the human body and fluidically connected to the chamber, and wherein the control unit is configured to calculate the length of movement Lᵢ according to the following formula:
$${L}_{i} = {L}_{i - 1} + ( {\prod }_{i} - {PR}_{i} ) \times \frac{Δ {t}^{2}}{S \times μ}$$
wherein
Lᵢ₋₁ is the length of movement over the cycle Cᵢ;
Πᵢ is the reference pressure on the cycle Cᵢ;
PRᵢ is the actual fluid pressure measured at the ejection cannula;
S is the equivalent section of the surface of the organ in contact with the fluid, and
μ is a constant.

14. The device according to claim 11, wherein the control unit is configured to generate, for each phase of ejection, a reference pressure curve according to the Franck-Starling law, the value of Πᵢ corresponding to the value of the pressure on said curve over the cycle Cᵢ.

15. The device according to claim 14, wherein the reference pressure curve according to the Franck-Starling law is identical for each phase of ejection.

16. The device according to claim 14, wherein the reference pressure curve according to the Franck-Starling law is redefined at each phase of ejection or redefined at regular time intervals according to blood pressure values measured at a pre-set frequency, at one end of the patient's body.

17. The device according to claim 11, wherein the control unit is configured to calculate, at each cycle Cᵢ, the reference pressure Πᵢ according to the following formula:
Πᵢ=(PMₛ−Kₛ·VEᵢ₋₁−Rₛ·Qᵢ₋₁)
wherein:
PMₛ is the maximum systolic pressure whose value is pre-set and/or regulated;
Kₛ is a coefficient comprised between 0.9 and 1.1;
VEᵢ₋₁ is the volume of fluid moved in the chamber during the phase of ejection in the previous cycle Cᵢ₋₁;
Rₛ is a coefficient comprised between 0.4 and 0.6;
Qᵢ₋₁ is the flow rate of fluid ejected from the chamber in the previous cycle Cᵢ₋₁.

18. The device according to claim 11, wherein the number N of cycles Cᵢ is between 45 and 55, and the duration Δt of each cycle Cᵢ is between 0.004 s and 0.006 s.

19. The device according to claim 11, wherein the control unit is configured to dissociate the phases of aspiration and the phases of ejection of the fluid such that during an artificial cardiac cycle, the volume aspirated during the aspiration phase is different from the volume ejected during phases of ejection.

20. The device according to claim 11, wherein:
the first cycle C₁ coincides with the natural systole, and starts at the instant when a QRS complex of an ECG electrocardiogramal measured on the patient is detected, or
the first cycle Cᵢ is delayed relative to the natural systole and starts after a delay comprised between 0.2 and 0.3 seconds from the instant when a QRS complex of an ECG electrocardiogramal measured on the patient is detected.
